(19) United States
(12) Patent Application Publication
(10) Pub. No.: US 2025/0001118 A1
(43) Pub. Date: Jan. 2, 2025

(54) DEVICE AND METHOD OF PRODUCING HYPOXIC AND HYPEROXIC GAS MIXTURES

(71) Applicant: Z+ HEALTH SRL, Noventa Vicentina (VI) (IT)

(72) Inventors: Alberto ZAMPERLA, Altavilla Vicentina (VI) (IT); Stuart John CLOUGH, Cambridge (GB)

(21) Appl. No.: 18/689,336

(22) PCT Filed: Sep. 7, 2022
(86) PCT No.: PCT/EP2022/074871

§ 371 Date: Mar. 5, 2024

(30) Foreign Application Priority Data
Sep. 7, 2021 (IT) 102021000023123

(51) Int. Cl. A61M 16/10; A61M 16/20; B01D 46/00; B01D 53/22

(52) U.S. Cl. CPC A61M 16/1005; A61M 16/105; A61M 16/1095; A61M 16/202; B01D 46/0027; B01D 53/22; A61M 2205/3334; A61M 2205/3368; A61M 2205/3606; A61M 2230/06; A61M 2230/20; B01D 2053/224; B01D 2256/10; B01D 2256/12; B01D 2257/102; B01D 2257/104; B01D 2259/4533

(57) ABSTRACT

A device (1) for providing hyperoxic/hypoxic breathable gas mixtures comprises a membrane tube assembly (2) for separating air into a high oxygen and low oxygen gas mixtures, a pressurized air inlet (2a), gas mixture outlets for permeate hyperoxic gas and retentate hypoxic gas mixture, means (6) for controlling the flow rate (F) of retentate gas mixture and means (7) for controlling the pressure (Pi) of the pressurized air fed to said inlet (2a), a control unit (100) connected to said means (6) for controlling the flow rate (F) of retentate gas and to the means (7) for controlling the pressure (Pi) of the pressurized air, valve means (21, 22) for regulating the flow of permeate and retentate gas mixtures from said outlets (2b, 2c).

### FIELD OF THE INVENTION

The present invention relates to a device and a method for producing hypoxic and/or hyperoxic gas mixtures. In particular, the device and the method according to the invention is suitable to provide a gas mixture having a predefined amount of oxygen, that is selected and controlled depending on the final use of the gas mixture.

For example, the gas mixture of the invention may be used to produce gas mixtures that may be administered for a user's rehabilitation therapy, or may be used for providing intermittent hyperoxic/hypoxic training (IHHT) to a subject.

In other words, the present invention relates to a device and a method to produce breathable air mixtures having different oxygen concentrations. Namely, the device and the method of the invention can generate hypoxic and hyperoxic air mixtures, i.e. breathable gas mixtures in which the oxygen concentrations are below or above the standard concentration of about 21% by volume.

### BACKGROUND OF THE INVENTION

It is known that breathing hypoxic air in the form of repeated short-term hypoxia exposures results in beneficial effects to the subject exposed.

Intermittent Hypoxic Training or Intermittent Hypoxic Treatment (IHT) is the common designation employed to indicate a treatment method using intermittent hypoxic exposure as the main therapeutic or training factor. A treatment session comprises a time interval breathing hypoxic (low oxygen) air, alternated with similar time intervals breathing ambient (normoxic) or hyperoxic air.

In case the alternation is between breathing hypoxic and normoxic air, the designation of the treatment method is usually IHT (i.e. Intermittent Hypoxic Training or Treatment), while in case the alternation is between breathing hypoxic and hyperoxic air, the designation of the treatment method is usually IHHT (i.e. Intermittent Hyperoxic/hypoxic Training or Treatment).

Standard practice is for the user to remain stationary while breathing hypoxic air via a hand-held mask. However, there is an interest in performing the treatment on an active subject, e.g. during physical exercise such as walking or running on a treadmill.

IHT and IHHT deprive the body of oxygen in predetermined time intervals. During treatment, oxygen saturation levels in the blood, heart rate, and blood pressure are monitored in order to safely reduce oxygen levels.

The core of the IHT/IHHT method is repeated reduction of blood oxygen to the individual hypoxia adaptation level intermingled with recovery intervals. The intermittent exposure to hypoxia during IHT/IHHT stimulates adaptation to altitude, and results in better circulation, improved mitochondrial function, increased tolerance to toxic chemicals, increased antioxidant production and reduced inflammation.

In other words, hypoxia adaptation can enhance physical and mental capacity of an individual. It should be noted that IHT/IHHT can be used for different application, including therapies and treatments used in rehabilitation for cardiovascular/pulmonary inquires, dementia or Alzheimer's diseases, metabolic disorders and sport medicine.

IHT/IHHT can also be used in contexts other than rehabilitation or curative therapy, for example in preventive and anti-aging medicine concerning wellbeing, or for treating stressor-related disorders such as burnout and exhaustion, or sleep disturbances.

IHT/IHHT can also be used in sport and fitness training, since one of the effects of this training is an increased tolerance to physical load, comprising an increased VO₂ max (i.e. increased maximal oxygen consumption, maximal oxygen uptake or maximal aerobic capacity) and increased exercise-until-exhaustion (ETE) time.

EP 1721629 discloses a list of advantages from hypoxic cycles, such as e.g. stimulating erythropoietin and haemoglobin production, exercising respiratory muscles and ventilation, producing hypotensive and vasodilative effects, reducing free-radical formation in the body and also increasing the body's antioxidant enzymatic capacity.

Breathing hypoxic air is disclosed in US2018333089 to be useful in sport training and as a method of contrasting loss of muscular force and mass with reduced risk and side effects compared to other methods.

Alternating hypoxic air with hyperoxic air, or Intermittent Hypoxic Hyperoxic Treatment (IHHT) is also known in the art. US2019134326 discloses a device and a method for providing alternated hypoxic and hyperoxic air mixtures to be supplied to a user for intermittent breathing the said mixtures in order to improve cognitive and functional abilities of the said user.

According to US'326 breathable air mixtures for an IHHT are generated with a low concentration of oxygen (9-21 vol. %) and a high oxygen concentration in the range 21-95 vol. %, by separation of ambient air into nitrogen and oxygen and afterward mixing them in a calculated ratio in order to provide the required oxygen concentration in a final air mixture that is fed to the user via a face mask.

Ambient air is separated into two flows of gas mixtures with PSA type process (e.g. zeolite adsorbents) fed to a plenum and subsequently combined together in a controlled way.

A problem of known methods for generating breathable air mixtures is that systems will produce oxygen levels above 96% by volume. Different adsorbents are normally used for oxygen concentration and nitrogen concentration.

The process is cyclic and results in the necessity of storing the product gas and exhaust gas, having high nitrogen concentration and high oxygen concentration, respectively before combining them into a mixture having the required oxygen concentration.

Another disadvantage is that the PSA process requires a stabilisation time on start-up. This can vary from several seconds to hours, depending on the design of the system. Response to changes of oxygen requirements by the user in known art may be too slow.

It is therefore an aim of the present invention to solve the above problem and provide an apparatus and a method for producing breathable air mixtures having different concentrations of oxygen wherein high flow rates of gas mixture and short times for changing the gas mixture composition are possible.

Apparatus and method are particularly useful for an intermittent hypoxic and hyperoxic treatment.

Said aim is reached by the present invention that provides a device according to claim 1 and a method according to claim 10. Preferred embodiments are recited in dependent claims.

### SUMMARY OF THE INVENTION

The present solution provides a device for providing hyperoxic/hypoxic gas mixtures comprising an oxygen content lower than in ambient air and an oxygen content increased with respect to ambient air.

The invention device comprises at least one membrane tube assembly that is configured to separate oxygen from nitrogen, an inlet for pressurized air and outlets for oxygen-rich permeate gas and nitrogen-rich retentate gas, and means for controlling the pressure and/or the flow rate of at least the nitrogen-rich air exiting the retentate outlet.

By controlling the pressure and/or the flow rate of the air flowing through the membrane tube assembly, the content of oxygen in the permeate gas and in the retentate gas can be controlled and set to a required predefined value. The gas mixture thus obtained can therefore be directly fed to the user of the device, without having to be mixed with ambient air or with the other gas exiting the membrane separator in order to reach the required oxygen content.

In other embodiments, the gas mixtures may also be fed to a user for other applications, requiring different levels of oxygen; the gas mixtures may be separately collected and stored.

For example, according to an embodiment, the gas mixture having the required predefined oxygen content may be compressed and stored in a suitable vessel for further subsequent use. An example of such use is compressed gas mixtures for professional or recreational underwater diving.

Accordingly, the device of the invention may be free from plenums or other devices for storing a volume of gas; in analogy, the invention method excludes the step of mixing together the permeate and the retentate gases exiting the separator device. The permeate or retentate gas mixture obtained according to the invention may be directly fed to the user through a dispensing device such as e.g. a face mask or a nasal cannula.

The result of subjecting a user to IHHT is to improve cognitive, physical and functional capabilities of the person by varying the blood oxygen saturation when the oxygen concentration in the gas mixture breathed by a subject is changed, and it includes intermittent feeding of hypoxic and hyperoxic gas mixtures via a face mask or other dispensing device, which is connected by a duct to the device according to the invention for hypoxic and hyperoxic gas mixtures.

In an embodiment, the device for providing hyperoxic/hypoxic gas mixtures according to the invention comprises:
at least one membrane tube assembly which separates pressurized air into a permeate gas mixture and a retentate gas mixture, said membrane comprising a pressurized air inlet and a first and a second gas mixture outlets,
a source of pressurized air for feeding pressurized air into said air inlet,
means of controlling the flow rate of said retentate gas mixture at said second outlet and means for controlling the pressure of the pressurized air fed at said inlet,
an electronic control unit connected to at least said means for controlling the flow rate of said retentate gas mixture at said second outlet and to said means for controlling the pressure of the pressurized air fed at said inlet,
valve means for regulating the flow of said permeate and retentate gas mixture exiting said outlets.

According to an embodiment, the device includes a dispensing device to provide a gas mixture having the required oxygen content to a user. The said valve means is operated for alternatively providing to said dispensing device either said permeate gas mixture through said first outlet or said retentate gas mixture through said second outlet.

It has to be noted that the gas delivery from the device is almost instantaneous, and the membrane tubes deliver the gas mixture with the predefined oxygen content as soon as they reach a suitable operating pressure.

For example, when the pressurisation rate is set at 4 bar per second and the operating pressure is set at 5 bar g, the device is able to deliver the gas mixture in less than two seconds from the air feed valve opening.

Advantageously, the device according to the invention excludes the step of mixing together the permeate and the retentate gases exiting the separator device. The permeate and retentate gas mixture obtained according to the invention may be separately fed to the user though a dispensing device such as e.g. a face mask or a nasal cannula in the absence of further treatment of the gas, contrary to the know art.

Thus, the gas mixtures obtained from the device and the method of the invention that are used for IHHT and medical or esthetical treatments are different and new with respect to the gas mixtures known in the prior art. In fact, the gas mixtures according to the invention are not mixed together to reach the required gas composition, i.e. they retain the composition typical of the retentate gas or permeate gas as produced by the tubular membrane assembly.

In such assembly, water vapour helium and hydrogen diffuse very quickly through the membrane wall; oxygen and carbon dioxide diffuse together at similar rates. The resulting gas mixtures exiting the membrane tubular assembly are a retentate nitrogen-rich mixture with very low content of water vapour, helium and carbon dioxide, and an oxygen rich gas mixture with a high content of water vapour, hydrogen, helium and carbon dioxide.

On the contrary, a gas mixture according the prior art is obtained by first separating air into a very high content oxygen gas mixture and a very high content nitrogen gas mixture, and subsequently re-mixing the obtained gas mixtures to reach the required oxygen level in the mixture. The resulting hypoxic gas mixture will inevitably contain in addition to nitrogen, also water vapour, hydrogen, helium and carbon dioxide that are present with oxygen in the permeate gas mixture that is added to the retentate gas mixture containing nitrogen.

According to an aspect, hyperoxic gas mixtures comprise an oxygen amount of at least 35% by volume, preferably in the range of 35% to 38%; hypoxic gas mixtures comprise an oxygen amount of less than 15%, preferably in the range of 10% to 13%.

Advantageously the hyperoxic and hypoxic gas mixtures provide respective oxygen percentages of two flows of gas mixtures, which do not need to be subsequently combined together in a controlled way, and can be delivered to the dispensing device, when present, or to separate storage containers, e.g. pressurized vessels or cylinders.

According to an aspect the means for controlling the flow rate of the retentate gas mixture at said second outlet comprises a back pressure regulator and/or a feed pressure regulator.

Back pressure regulators and pressure regulators are devices well known in the art (see for example https://en.wikipedia.org/wiki/Pressure_regulator).

It has to be noted that Back Pressure Regulators (BPR) are known in the art as devices that maintain a defined pressure upstream of their own inlet. When the fluid pressure at the inlet of the back pressure regulator exceeds a predetermined setpoint, the regulator opens to relieve the excess pressure.

On the contrary, Pressure Regulators or Pressure Reducing Regulators (PR) are known in the art as devices that maintains a defined pressure downstream of their own outlet. When the fluid pressure at the outlet of the pressure regulator exceeds a predetermined setpoint, the regulator closes to relieve the excess pressure.

In other words, Pressure Regulators reduce a higher supply pressure down to a regulated lower pressure at the outlet, maintaining a desired downstream pressure. Back pressure regulators work the opposite way. They regulate the pressure at the inlet by opening up only as much as necessary to maintain the desired upstream pressure. According to an aspect, the means for controlling the pressure of the pressurized air fed at said inlet comprises a feed pressure regulator.

According to an aspect, the invention device comprises means for controlling the temperature of the pressurized air fed at said inlet of said tube membrane, which comprise an air cooler.

Advantageously, the presence of above cited control means for regulating several parameters of the gas mixtures, allows acting directly on the oxygen percentages of the gas mixtures to be delivered by e.g. controlling the flow rate of the air through the membrane tubes.

According to an aspect, in case the device is used to directly feed the gas mixture to a user, in particular when used for IHHT or sport training, the device may comprise means for monitoring at least one body parameter. According to this aspect, said means for monitoring at least one body parameter comprise at least a pulse oximeter.

According to an aspect, the device comprises at least one filtration unit for purifying said pressurized air before feeding it to said inlet.

The present invention is further directed to a method according to claim 10 of producing hypoxic and hyperoxic breathable gas mixtures with a device according to any claim 1, comprising the steps of feeding pressurized air to a membrane tube assembly containing at least one hollow fiber, preferably a plurality of hollow fibers, said membrane tube assembly being configured to provide a first flow of a hyperoxic gas mixture and a second flow of hypoxic gas mixture, wherein the hypoxic gas mixture is the retentate gas mixture exiting said membrane tube assembly, further comprising the step of regulating the oxygen content of said hyperoxic gas mixture and of said hypoxic gas mixture by controlling the pressure and/or the flow rate of said hypoxic gas mixture exiting from said membrane tube assembly through a retentate outlet.

According to an aspect, each gas mixture exiting said membrane tube assembly is further used as is, e.g. it is fed to a dispensing device without being mixed with ambient air or with the other gas mixture exiting the membrane tube assembly.

The present invention is further directed to a method of performing intermittent hyperoxic/hypoxic training (IHHT) by operating a device according to any claim 1 to 9, said device comprising a gas mixture dispensing device, means for monitoring at least one body parameter of the user, the method comprising the steps of:
feeding pressurized air to an air inlet of said tube membrane,
detecting at least one body parameter with said monitoring means,
directly or indirectly calculating the oxygen saturation level SpO₂ from said detected body parameter and comparing it with a maximum reference level SPO_{2 r-max} and with minimum reference level SPO_{2 r-min},
switching valve means for alternatively providing to a dispensing device said hyperoxic gas mixture in case said oxygen saturation level SpO₂ is lower than said minimum reference level SpO₂ r-min, or said hypoxic gas mixture in case said oxygen saturation level SpO₂ is higher than said maximum reference level SpO₂ r-max,
   wherein said step of switching said operated valve means results in alternatively providing a hypoxic or a hyperoxic gas mixture to said dispensing device.

According to an aspect, the maximum reference level of oxygen saturation SpO_{2 r-max} is in the range of 98% to 100% by volume, and the minimum reference level of oxygen saturation (SpO_{2 r-min}) is a value comprised in the range of 84% to 86% by volume.

According to an aspect, the at least one body parameter comprise oxygen saturation level (SpO₂) and/or a heart rate (BPM).

The invention also refers to a breathable mixture as obtained by a method and a device according to the invention, for use in medical treatment.

The invention also relates to a method of medical or esthetical treatment that comprises administering an effective amount of a hypoxic or hyperoxic gas mixture as obtainable by a device according to any claim 1 to 9.

### DESCRIPTION OF THE FIGURES

Exemplary embodiments of the present invention are now described in greater detail with reference to the accompanying drawings provided by way of non-limiting example, wherein:

FIG. 1 is a block diagram of a possible embodiment of the device 1 according to the invention;

FIG. 2 is a schematic view of an individual hollow fibre membrane according used in the invention;

FIG. 3 is a schematic view of the membrane tube assembly 2 according to the invention;

FIG. 4 is a a graph showing oxygen saturation (SpO2), pulse rate (bpm) and the IHHT delivery gas oxygen percentage during an exemplary working cycle of the device according to the invention;

FIG. 5 is a block diagram of an embodiment of the invention device for wellness applications;

FIG. 6 is a block diagram of a possible embodiment of the device according to the invention for sport training applications;

FIG. 7 is a block diagram of a further embodiment of the device for wellness applications;

FIG. 8A is a screen of the user interface showing the System Overview screen;

FIG. 8B is a screen of the user interface showing the User Graphics;

FIG. 8C is a screen of the user interface showing the Engineering screen.

### DETAILED DESCRIPTION OF THE INVENTION

With reference to figures, an exemplary device 1 for providing hyperoxic/hypoxic gas mixtures comprises at least one membrane tube assembly 2 which separates pressurized air into two gas mixtures, a first gas mixture outlet for permeate hyperoxic gas mixture 2b and a second gas mixture outlet 2c for retentate hypoxic gas mixture.

The pressurized air is provided by a source of pressurized air 3. In general, the source of pressurized air may be any of an air compressor, a pressurized vessel such as a pressurized air cylinder, or a ring main compressed air piping. Typically, air may be medical grade air. Pressure control means 7 for controlling the pressure of the pressurized air fed to said membrane tube assembly 2 are provided upstream of tubular membrane assembly 2. Device 1 comprises a dispensing device 5 to deliver the required hyperoxic/hypoxic gas mixture to a user, and means 6 for controlling the flow rate F of the retentate gas mixture exiting assembly 2.

An electronic control unit 100 is connected to at least means 6 for controlling the flow rate of the retentate gas mixture exiting the membrane tube assembly 2 and to means 7 for controlling the pressure of the pressurized air fed to the membrane tube assembly 2. By selecting a proper value of the air pressure and of the flow rate, the desired composition of the permeate and/or retentate gas mixtures may be obtained.

The device 1 comprises at least one valve means 21, 22 for regulating the flow of the permeate and retentate gas mixtures flowing from their outlets 2b, 2c.

The membrane tube assembly 2 comprising individual hollow fibres 200, is capable of separating air components, thus producing gas mixtures in the form of oxygen enriched air and oxygen depleted air. Suitable membrane tube assemblies are commercially available, e.g. as nitrogen generators from Parker filtration and separation BV, under the name Hifluxx®.

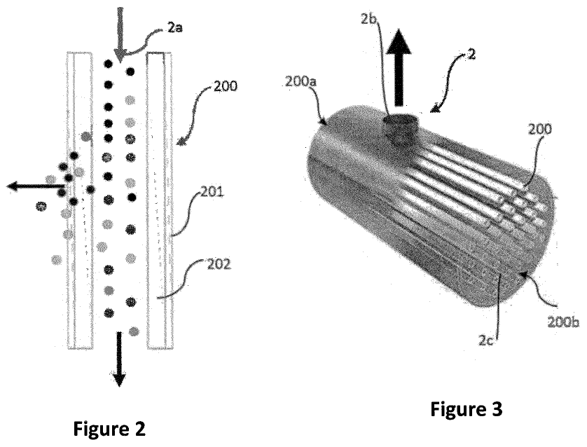

In particular, the membrane tube assembly 2, comprises bundles of hollow fibres 200—contained within a tube assembly 2. In a preferred embodiment schematically shown in FIG. 2, the hollow fibres 200 have a spun permeable membrane 202 that selectively separate pressurized air into a low oxygen mixture (retentate) and an oxygen enriched mixture (permeate), and an outer tube 201 which provides the structural strength to membrane 202.

The membrane assembly 2 comprises a pressurized air inlet 2a and first and second gas mixture outputs 2b, 2c; a source of pressurized air 3 provides pressurized air into the air inlet 2a of membrane tube assembly 2. In a possible embodiment, the source of pressurized air is a compressor 3, such as a variable speed screw compressor 3. A suitable working pressure is in the range of 4 bar g to 8 bar g.

It has to be noted that here and in the following the pressure value is given in gauge pressure (bar g), i.e. pressure in bars above ambient or atmospheric pressure (1 bar).

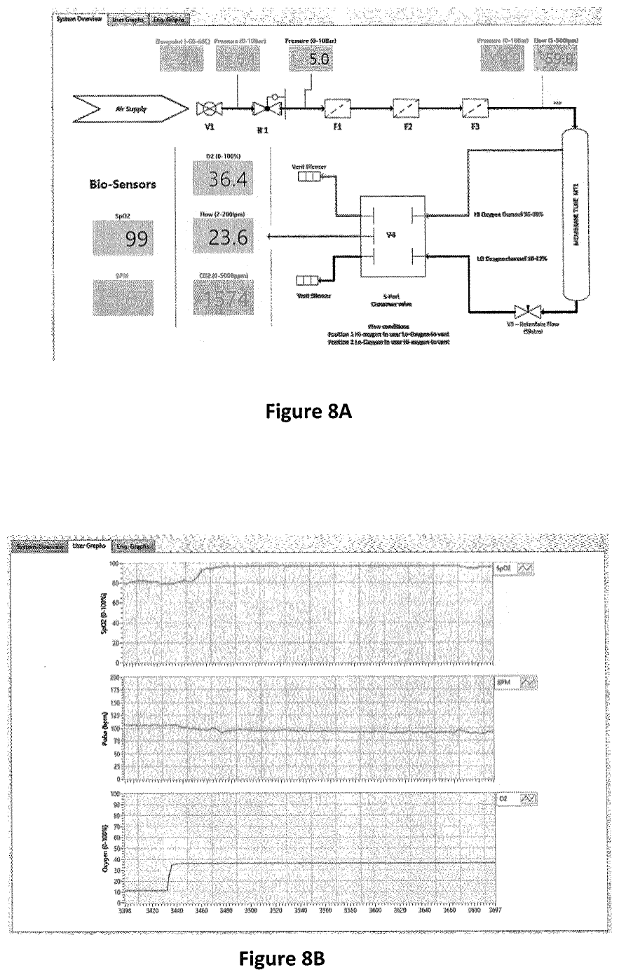

In a further possible embodiment, compressor 3 may be a non-modulating compressor 3, used in combination with an air receiver tank 32. In a possible embodiment shown in FIG. 7 device 1 comprises a source of pressurized air 3 which provides air from a facility ring main 3.

The oxygen enriched mixture (permeate) and the low oxygen mixture (retentate) exit the membrane tube assembly 2 through the first and second outlet 2b, 2c respectively, to be then delivered through respective channels 20b, 20c to a dispensing device 5 or to a collecting device. According to a possible embodiment, the dispensing device 5 comprises one of a nasal cannula or a face mask.

According to a possible embodiment, the device 1 may comprise a second compressor, not shown, positioned downstream of valve means 21, 22.

The second compressor will feed pressurized hypoxic or hyperoxic gas mixture into a reservoir, not shown, instead of the dispensing device 5, wherein it is stored for future use.

For example, the reservoir can be a cylinder or a scuba tank used in underwater activities.

As schematically shown in FIG. 2, the hollow fibre 200 consists, in a way known in the art, of a permeable structure 202 with an ultrathin cover layer 201. According to a possible embodiment, the hollow fibres 200 are held in a module 2 which is a metal or plastic tube. Preferably, the ends 200a, 200b of fibres 200 are bound, e.g. glued, together and are fixed to the tube so that pressurized air fed to the air inlet 2a, enters the fibres 200.

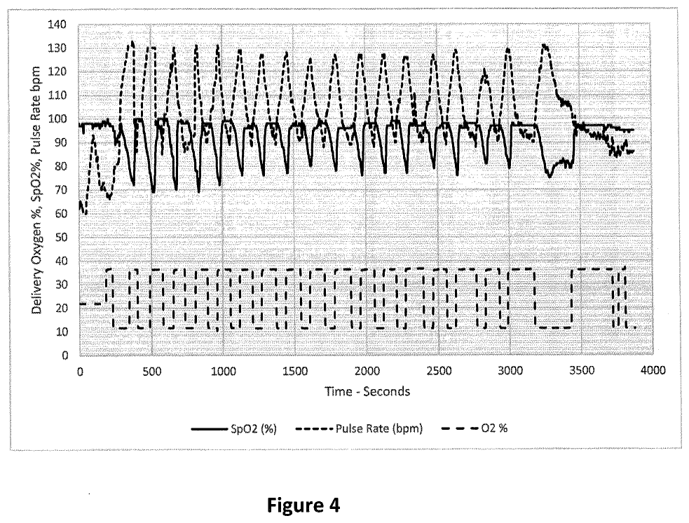

As schematically represented in FIGS. 2 and 3, membrane fibres 200 allow oxygen and water molecules of the pressurized air to permeate through the wall of the membrane fibers in tube assembly 2 faster than the nitrogen molecules, such that most of water and oxygen molecules are discharged from the membrane tube assembly 2 as a permeate product, through first outlet 2b. Most of nitrogen molecules remain inside the fibre and exit at the second outlet 2c of the membrane tube assembly 2 as a retentate product.

As will be disclosed in detail, the pressure Pi and temperature T at which the compressed air enters the fibres 200 of the membrane tube assembly 2, and the time the air remains inside the membrane tube assembly 2, determines the oxygen content in the hyperoxic and hypoxic gas mixtures at the first and second outlets 2b, 2c.

For these reasons, the device 1 according to the invention comprises means 6 for controlling and regulating the flow rate F of the gas mixture at the second outlet 2c and means 7 for at least controlling the pressure Pi of the pressurized air fed at said inlet 2a.

An electronic control unit 100 is connected at least to these means 6, 7 for controlling the flow rate F of the retentate gas mixture at the second outlet 2c and the pressure Pi of the pressurized air fed at the inlet 2a.

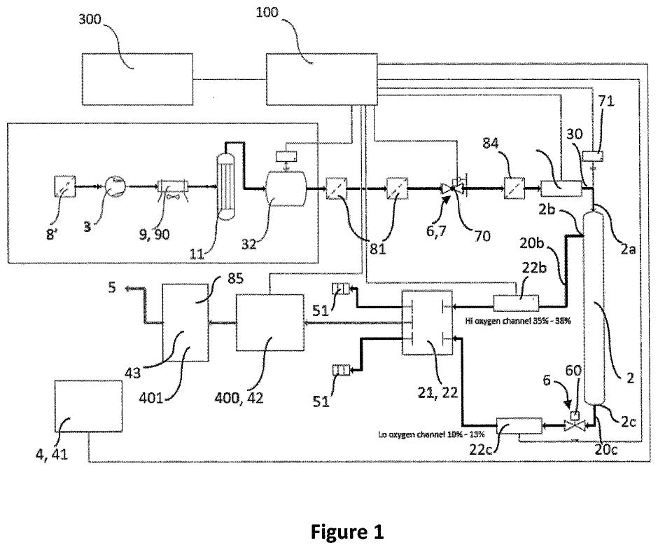

According to a possible embodiment shown in FIG. 1, the means 7 for controlling the pressure Pi of the pressurized air fed at the inlet 2a, and the means 6 for controlling and regulating the flow rate F of the retentate gas mixture at said second outlet 2c, comprise a feed pressure regulator 70.

In particular, the feed pressure regulator 70 is positioned along the pressurized air feed line 30, downstream of the source of pressurized air 3 and upstream of the inlet 2a of the membrane tube assembly 2.

In a possible embodiment, the device 1 further comprises a pressure sensor 71, preferably positioned along the pressurized air feed line 30, downstream of the feed pressure regulator 70 and upstream of the inlet 2a of the membrane tube assembly 2.

The pressure sensor 71 is connected to said control unit 100, for monitoring the current value of the pressure Pi of the pressurized air fed at the inlet 2a.

In this way, according to the pressure value detected by the pressure sensor 71, the pressure regulator 70 can be operated to increase or decrease the pressure value of the pressurized air at its own outlet.

In a possible embodiment, when the pressure sensor 71 detects a gas mixture pressure value that exceeds a predetermined setpoint, the pressure regulator 70 can close to relieve the excess pressure, maintaining a desired downstream pressure.

In a possible embodiment, means 6 for controlling the flow rate F of the retentate gas mixture at the second outlet 2c, further comprise a back pressure regulator 60. The back pressure regulator 60 is preferably positioned downstream of the second outlet 2c and upstream of the dispending device 5 and of the valve means 21, 22.

In this way, the back pressure regulator 60 maintains a defined pressure upstream of its own inlet. When the gas mixture pressure at the second outlet 2c exceeds a predetermined setpoint, the regulator 60 opens to relieve the excess pressure.

According to a possible embodiment, the device 1 comprises means 9 for controlling the temperature T of the pressurized air fed at the inlet 2a of the tube membrane 2, which may comprise an air cooler 90.

In a possible embodiment, said means 9 for controlling the temperature T of the pressurized air fed at the inlet 2a of the tube membrane 2, further comprise at least one temperature sensor 91, for detecting the temperature of the air fed at the inlet 2a of the tube membrane 2, which can also be suitable for fire detection. Preferably, the temperature sensor is connected to the electronic control unit 100.

Preferably, the air cooler 90 is positioned along the pressurized air feed line 30, downstream of the source of pressurized air 3 and upstream of the pressurized air inlet 2a, more preferably upstream of the feed pressure regulator 70.

In a possible embodiment according to the invention, the membrane tube assembly 2 operates at air inlet pressures (Pi) ranging from 4 bar g to 13 bar g.

The amount of hyperoxic/hypoxic gas mixtures that can be produced at the outlets 2b, 2c of the membrane tube assembly 2 increases proportionally with the value of the air inlet pressure Pi.

As above mentioned, the performance of the tube membrane 2 in terms of oxygen percentage in the hyperoxic/hypoxic gas mixtures, depends on several parameters, among which the pressure value Pi of the pressurized air at the inlet 2a of the tube membrane, the flow rate F of the retentate gas mixture at the second outlet 2c, and the temperature T of the pressurized air fed at the inlet 2a of the tube membrane 2.

In more detail, by varying the flow rate F of the retentate gas mixture at the second outlet 2c, the oxygen content in the hyperoxic/hypoxic gas mixtures at the outlets 2b, 2c will change.

In particular, with decreasing the flow rate F of the retentate gas mixture at the second outlet 2c, the residence time of the air in the tube membrane 2 will increase and as a result the oxygen content in the hypoxic gas mixture at the second outlet 2c will be lowered and the oxygen content in the hyperoxic gas mixture at the first outlet 2b will be increased.

In other words, the percentage content of oxygen in the hyperoxic/hypoxic mixtures can be adjusted by tuning the flow rate F of the retentate gas mixture at the second outlet 2c.

The tubes are sized to suit the flows and gas mixtures required. Flow rate, pressure and preferably also temperature are adjusted and set according to the required composition of retentate and permeate in a way know per se in the art. In fact, producers of the membrane tubes usually provide relevant charts showing changes of composition as a function of at least the flow rate and pressure. Furthermore, the performance of the tube membrane 2 is influenced by the temperature T at which the membrane tube assembly 2 operates: the membrane tube assembly 2 operates optimally at a temperature T comprised in the range of 2° C. to 50° C. and increasing temperature will result in higher pressurized air consumption.

Furthermore, increasing the pressure inside hollow fibres 200 of membrane tube assembly 2 results in increasing the capacity of the device: the pressurized air consumption increases proportionally.

The device 1 according to the invention, comprises valve means 21, 22 for regulating the flow of permeate gas mixture (hyperoxic gas mixture) flowing from the first output 2b to the dispensing device 5, and the flow of the retentate gas mixture (hypoxic gas mixture) flowing from the second output 2c to the dispensing device 5.

In a possible embodiment, said valve means is a single valve comprising two different paths 21, 22 for the permeate gas mixture and retentate gas mixture respectively.

The valve means 21, 22 is operated such that the hyperoxic/hypoxic gas mixtures are alternatively and separately delivered to said dispensing device 5 which is positioned downstream of the valve means 21, 22.

In a further possible embodiment, the valve means comprise a first valve means 21 for regulating the permeate gas mixture (hyperoxic gas mixture) flowing from the first output 2b to the dispensing device 5, and second valve means 22 for regulating the retentate gas mixture (hypoxic gas mixture) flowing from the second output 2c to the dispensing device 5.

The first and second valve means 21, 22 are alternatively operated, such that the hyperoxic/hypoxic gas mixtures are alternatively delivered to dispensing device 5 which is positioned downstream of the valve means 21, 22.

In a possible embodiment, shown in FIG. 1 the device 1 comprises a vent 51 connected to the valve means 21, 22.

For example, in a possible embodiment shown in FIGS. 1 and 7 the valve means is a five-way valve, for example a 5-port crossover valve, comprising two different paths (outlets) 21, 22 for the permeate gas mixture and retentate gas mixture respectively.

According to this embodiment, the valve 21, 22 has two inlets for the feeding of the respective gas mixtures from the outlets 2b, 2c, and three outlets.

One of the three outlets of the five way valve 21, 22 is connected to the dispensing device 5, and two of the three outlets of the five way valve 21, 22 are connected to a vent 51.

In a possible configuration of the device according to the invention, the five way valve means 21, 22 is alternatively operated such that, when in operation, one outlet of the valve means 21, 22 fluidically connects the respective outlet 2b, 2c of the membrane tube assembly 2 with the dispensing device 5, while one outlet connects the respective outlet 2c, 2b of the membrane tube assembly 2 with vent 51. In this way, the hypoxic or the hyperoxic mixtures are alternatively fed to the dispensing device 5, and the device 1 can be used for intermittent hyperoxic/hypoxic training (IHHT).

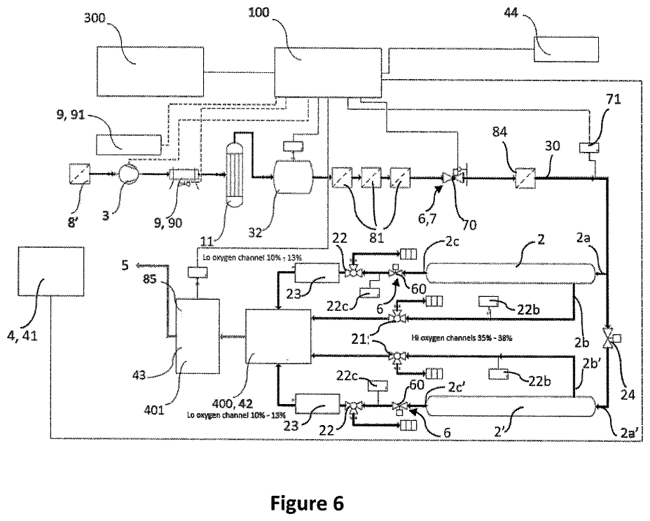

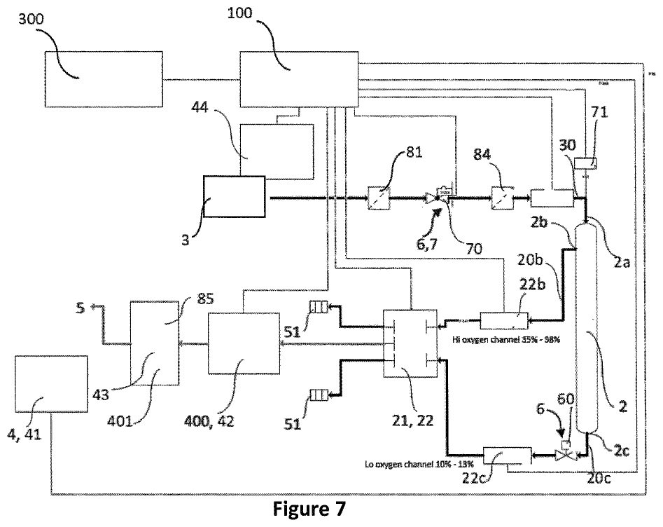

In a possible embodiment, shown in FIGS. 5 and 6, the valve means 21, 22 comprise three-way valves.

According to this embodiment, each valve 21, 22 has one inlet for feeding of the respective gas mixtures from the outlets 2b, 2c, and two outlets. One of the two outlets of a three way valve 21, 22 is connected to the dispensing device 5, and one of the two outlets of the three way valve 21, 22 is connected to buffer 51.

In a preferred configuration of the device according to the invention, the valve means 21, 22 are alternatively operated such that, when in operation, one valve means 21, 22 fluidically connects the respective outlet 2b, 2c of the membrane tube assembly 2 with the dispensing device 5, while the other valve means 22, 21 connects the respective outlet 2c, 2b of the membrane tube assembly 2 with the vent buffer 51.

In this way, the hypoxic or the hyperoxic mixtures are alternatively fed to the dispensing device 5, and the device 1 can be used for intermittent hyperoxic/hypoxic training (IHHT).

In a possible embodiment the hyperoxic gas mixture delivered to the dispensing device 5 comprises an oxygen content of at least 35%, preferably between 35%-38%, and the hypoxic gas mixture delivered to the dispensing device 5 comprises an oxygen content less than 15%, preferably between 10-13%.

According to a possible embodiment, the oxygen content of the hyperoxic and hypoxic gas mixtures at the outlets 2b and 2c are measured by two respective sensors 22b, 22c positioned downstream of said outlets 2b, 2c and upstream of said dispensing device 5.

Preferably, said sensors 22b, 22c for measuring the oxygen percentage of the gas mixtures are connected to control unit 100.

According to a possible embodiment, the device 1 comprises a humidifier 23 positioned downstream of the outlet 2c of the hypoxic mixture.

The above mentioned oxygen percentages can be varied, as above discussed, by varying several parameters, among which the pressure value Pi of the pressurized air at the inlet 2a of the tube membrane, the flow rate F of the retentate gas mixture at the second outlet 2c, and the temperature T of the pressurized air fed at the inlet 2a of the tube membrane 2.

In a possible embodiment according to the invention, the device 1 comprises means 4 for monitoring at least one body parameter VPₘ, for example a pulse oximeter 41 and/or a capnograph 42 to monitor the concentration of carbon dioxide.

For example, the body parameters VPₘ which can be monitored comprise oxygen saturation level (SpO2) and/or a heart rate (BPM) and/or the concentration or partial pressure of carbon dioxide (CO₂) in the gas mixtures. In a possible embodiment, the device 1 comprises at least one filtration unit 8′ for purifying the air before feeding it to the source of pressurized air 3.

In a possible embodiment, the device 1 comprises at least one filtration unit 81 for purifying the pressurized air before feeding it to said inlet 2a. Preferably, the at least one filtration unit 81 is positioned along the air feeding line 30, downstream of the source of pressurized air 3 and upstream of the air inlet 2a. In a possible configuration, as shown in FIG. 1 the device 1 comprise one or more of the following filters: a coarse and fine coalescing filter to remove particles and liquids from pressurized air to be fed to the membrane 2, a carbon absorber of the bed type to remove hydrocarbons and oil fumes from pressurized air, and an activated carbon bed dust filter to prevent carbon dust carry-over.

Furthermore, the device 1 can comprise a further viral, bacterial and bacteriophage filter 84 as shown in FIG. 1, positioned upstream of the air inlet 2a and downstream of the above described filter assembly 81.

An exemplary filter 84 comprises a polypropylene prefilter layer and an inherently hydrophobic expanded PTFE membrane, for ensuring the removal of all airborne bacteria, viruses and bacteriophage.

In a possible embodiment, the device comprises a dryer/evaporation module 11 to create a non-condensing feed-air supply for the membrane tube assembly. Preferably, the dryer/evaporation module 11 is positioned along the air feeding line 30, downstream of the source of pressurized air 3 and upstream of the air inlet 2a, preferably upstream of the filtration unit 81.

The dryer/evaporation module is suitable to lower the dewpoint of the compressed air stream fed to the membrane tube assembly 2 such that the pressure dew point of the compressed air needs is at least 5° C. lower than the lowest ambient temperature to be expected, and remove the condensate that is formed.

According to an aspect, the device 1 comprises at least one sensor module 400, positioned downstream of the valve means 21, 22 and upstream of the dispensing device 5. Preferably this sensor module 400 is connected to the control unit 100. Sensor module 400 comprises a plurality of sensors for monitoring several parameters of the gas mixture which is fed from the operated valve means 21, 22 to the dispensing device 5. Preferably, the plurality of sensors comprise at least one of a capnograph, a sensor for measuring the oxygen content of the gas mixture, a flowmeter, a temperature sensor and a hygrometer.

According to a possible embodiment, the device 1 can comprise other sensors for monitoring different operating conditions, such as an ambient air quality sensor 44.

In a possible embodiment, device 1 comprises a delivery gas breathing bag 401 positioned downstream of the valve means 21, 22 and upstream of the dispensing device 5, wherein the hyperoxic/hypoxic mixture is stored before being delivered to the user.

According to an embodiment, the delivery gas breathing bag 401 comprises a UV filtration unit 85, or a further filtration unit similar to above disclosed filter unit 81.

The delivery gas breathing bag 401 can further comprise a level/volume sensor 43 connected to control unit 100, for monitoring the volume of the mixture inside the breathing bag 401.

According to a possible embodiment, represented in FIG. 6, the device 1 can comprise two membrane tube assemblies 2, 2′, connected in parallel between the pressurized air feed line 30 and the breathing bag 401. This configuration of the device 1 can be used in IHHT sport training applications.

Both the membrane tube assemblies 2, 2′ have a pressurized air inlet 2a, 2a′ and a first and a second gas mixture outputs 2b, 2c; 2b′, 2c′ as above described. According to this configuration, represented in FIG. 6, the device 1 comprises a warm up/training mode valve 24, positioned on the pressurized air feed line, upstream of the air inlets 2a, 2a′ of the two membrane tube assemblies 2, 2′.

The valve 24 can be operated for switching from a first operating mode which is a warm up mode, wherein the pressurized air is delivered to the first membrane tube assembly 2, and a second mode which is a training mode, wherein the pressurized air is delivered to both the first and the second membrane tube assemblies 2, 2′.

This results in that in the training mode a larger volume of pressurized air is supplied to the device 1 with respect to the warm up mode, such that a larger volume of hyperoxic/hypoxic gas mixture can be produced in this training mode, to be delivered to a user.

The present invention is further related to a method for operating a device 1 according to the invention comprising means 4 for monitoring at least one body parameter, for performing intermittent hyperoxic/hypoxic training (IHHT).

The method according to the invention comprises continuously feeding pressurized air to the air inlet 2a of the tube membrane assembly 2. Tube membrane 2 continuously separates the pressurized air fed to the inlet 2a, whereby two streams of hypoxic and hyperoxic gas mixtures are continuously provided.

The method further comprises the step of detecting at least one body parameter VPₘ by using monitoring means 4 for monitoring at least one body parameter, preferably at least oxygen saturation level SpO₂ and/or a heart rate BPM and/or the concentration or partial pressure of carbon dioxide CO₂ in the gas mixtures.

As above mentioned, the means 4 for monitoring the body paramenters VPₘ can comprise an oximeter 41, and a capnograph 42. Following the detection of the least one body parameter VPₘ, the method comprises the step of directly or indirectly calculating the oxygen saturation level SpO₂ from the detected body parameter VPₘ and comparing it with a maximum reference level SpO_{2 r,max} and with minimum reference level SPO_{2 r,min}.

According to a possible embodiment, the maximum reference level SpO₂ r,max and with minimum reference level SPO_{2 r,min} can be stored in the control unit 100, and the measured value of oxygen saturation level SpO₂ is compared with said maximum and minimum level.

Preferably, the maximum reference level of oxygen saturation SpO_{2 r,max} is a percentage value comprised between 98% and 100%, and the minimum reference level of oxygen saturation SpO_{2 r,min} is a percentage value comprised between 84% and 86%.

The method further comprises the step of switching the operated valve means 21, 22 such that the first valve means 21 is operated for providing the hyperoxic gas mixture to the dispensing device 5 in case the oxygen saturation level SpO₂ is lower than the minimum reference level SpO_{2 r,min}, and the second valve means 22 is operated for providing the hypoxic gas mixture to the dispensing device 5 in case in case said oxygen saturation level SpO₂ is higher than the maximum reference level SPO_{2 r,max}.

In this manner, the step of switching the operated valve means 21, 22 results in alternatively providing a hypoxic or a hyperoxic gas mixture to the dispensing device 5.

It has to be noted that when in operation, one valve means 21, 22 fluidically connects the respective outlet 2b, 2c of the membrane tube assembly 2 with the dispensing device 5, while the other valve means 22, 21 connects the respective outlet 2c, 2b of the membrane tube assembly 2 with a vent 51, which discharges the gas mixture to the atmosphere.

In this way, the hypoxic or the hyperoxic mixtures are alternatively fed to the dispensing device 5, and the device 1 can be used for intermittent hyperoxic/hypoxic training (IHHT).

In a possible embodiment, the changeover of the operating condition of valve 21, 22 is triggered by the comparison of the measured value of the oxygen saturation level SpO₂ with the maximum and minimum reference levels SpO₂ r,max and SpO₂ r,min which can be stored in the control unit 100.

In particular, in a possible embodiment, when the user is breathing a hyperoxic gas mixture having 35%-38% oxygen, delivered through said first valve means 21 to said dispensing device 5, the SpO₂ value is normally comprised between 98-100%, i.e. it is approximatively at maximum reference level of oxygen saturation SpO_{2 r,max}.

This condition wherein the oxygen saturation level SpO₂ reaches said maximum reference level SpO_{2 r,max} determines the changeover of the operated valve means.

This results in that valve means 21, 22 is operated for delivering the hyperoxic gas mixture to the vent 51, and for delivering the hypoxic gas mixture to the dispensing device 5.

Following this changeover, the user is breathing a hypoxic gas mixture having 10%-13% oxygen delivered through said second valve means 22 to said venting device 5, and the measured SpO₂ value gradually falls.

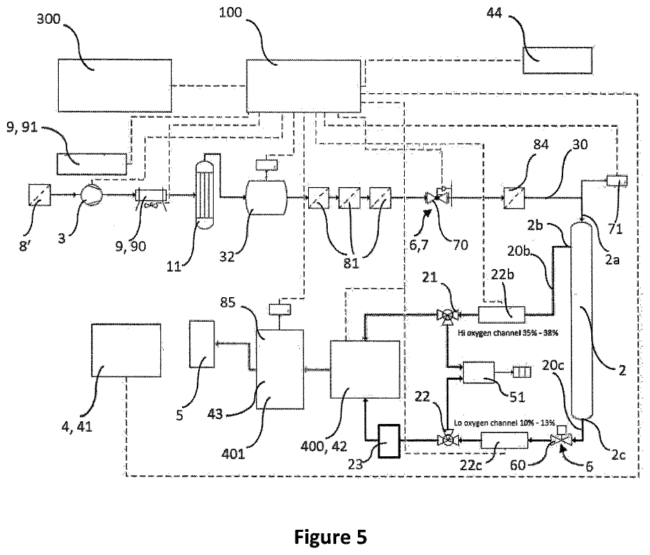

As shown in FIG. 4, this process provides a hysteresis: the effects of breathing an hypoxic gas mixture having reduced oxygen level, results in that the user's SpO₂ falls through 85%, i.e. it is approximatively at minimum reference level of oxygen saturation SpO_{2 r,min}.

This condition wherein the oxygen saturation level SpO₂ reaches said minimum reference level SpO_{2 r,min} determines the changeover of the operated valve means.

This results in that valve means 21, 22 is operated for delivering the hyperoxic gas mixture to dispensing device 5, and for delivering the hypoxic gas mixture to vent 51.

Following this change of the operating valve, the user is breathing a hyperoxic gas mixture having 35%-38% oxygen delivered through valve means 21, 22 to said dispensing device 5, and the measured SpO₂ value gradually rises back. As the oxygen enriched air is breathed by the user, the SpO₂ will quickly return to 98-100%. Two or three minutes of recovery time is then followed by another changeover to a low oxygen cycle.

According to a possible embodiment the device 1 is provided with a user interface 300, which is accessible to the user. In particular, by means of this user interface 300 the user can set several parameters for the delivery of gas mixtures, including the duration of a low oxygen cycle and/or of an increased oxygen cycle, and/or the overall duration of an intermittent hyperoxic/hypoxic training (IHHT).

The user interface 300 can comprise different selectable screens.

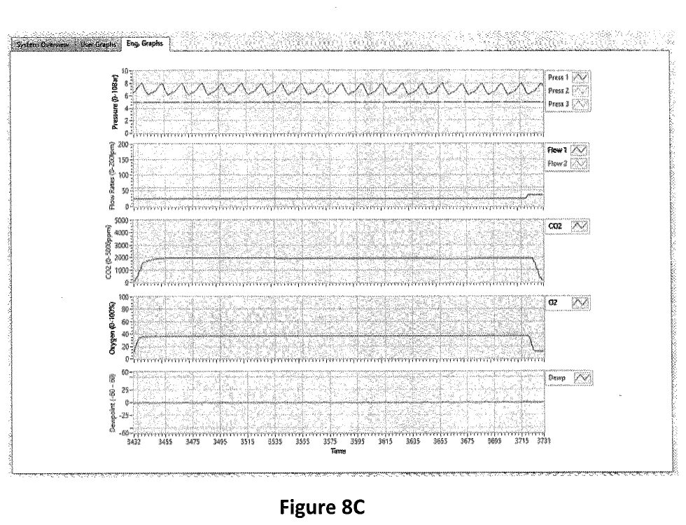

Exemplary embodiments are shown in FIGS. 8A, 8B and 8C:
System Overview screen (FIG. 8A), which shows real time values for system parameters and the body parameters detected by the sensors, such as pulse rate and SpO2;
User graphics screen (FIG. 8B), which shows the real time graphs of body parameters recorder for the critical values of SpO2, Pulse Rate, and percentage value of oxygen delivered;
Engineering screen (FIG. 8C) which shows in real time the graphs for the system parameters, such as pressures, gas levels and dew points.

In a possible embodiment, the oxygen content (percentage) in the hyperoxic/hypoxic gas mixtures is a function of at least the pressure (Pi) applied to the pressurized air injected into said tube membrane 2 and/or of at least the flow rate of the retentate gas mixture F defined at the second outlet 2c of the membrane tube assembly 2 and/or of at least the temperature T applied to the pressurized air injected into said tube membrane 2.

For these reasons, in a possible embodiment of the method according to the invention the oxygen percentage in the hyperoxic/hypoxic mixtures can be controlled by means of at least the means 6 for controlling the flow rate of the retentate gas mixture (F) at the second outlet 2c, which comprise a back pressure regulator 60 and/or a feed pressure regulator 70.

The back pressure regulator 60 maintains a defined pressure upstream of its own inlet. When the gas mixture pressure at the second outlet 2c exceeds a predetermined setpoint, the regulator 60 opens to relieve the excess pressure. Furthermore, the oxygen content in the hyperoxic/hypoxic mixtures can be controlled by means of at least the means 7 for controlling the pressure Pi of the pressurized air fed at the inlet 2a, which comprise a feed pressure regulator 70. Furthermore, the oxygen percentage in the hyperoxic/hypoxic mixtures can be controlled by means of at least means 9 for controlling the temperature T of the pressurized air fed at the inlet 2a.

1. A device (1) for providing hyperoxic/hypoxic breathable gas mixtures comprising
at least one membrane tube assembly (2) for separating air into a permeate gas mixture and a retentate gas mixture, said membrane tube assembly (2) comprising a pressurized air inlet (2a), a first gas mixture outlet for permeate hyperoxic gas mixture (2b) and a second gas mixture outlet for retentate hypoxic gas mixture (2c),
a source of pressurized air (3) for feeding pressurized air to said air inlet (2a),
means (6) for controlling a flow rate (F) of said retentate gas hypoxic mixture exiting said second outlet (2c) and means (7) for controlling pressure (Pᵢ) of the pressurized air fed to said inlet (2a),
a control unit (100) connected at least to said means (6) for controlling the flow rate (F) of said retentate hypoxic gas mixture exiting said second outlet (2c) and to the means (7) for controlling the pressure (Pᵢ) of the pressurized air fed to said inlet (2a), and
valve means (21, 22) for regulating said permeate and retentate gas mixtures flowing from said outlets (2b, 2c).

2. The device (1) according to claim 1, further comprising a dispensing device (5) for alternatively dispensing said first or second gas mixture, wherein said valve means (21, 22) is connected to said dispensing device operated for alternatively providing said permeate gas mixture or said retentate gas mixture flowing from said outlets (2b, 2c) to said dispensing device (5).

3. The device according to claim 1, wherein said permeate hyperoxic gas mixture comprises an oxygen percentage of at least 35%, and said retentate hypoxic gas mixture comprises an oxygen percentage less than 15.

4. The device (1) according to claim 1, wherein said means (6) for controlling the flow rate of the retentate gas mixture (F) at said second outlet (2c) comprise a back pressure regulator (60) and/or a feed pressure regulator (70).

5. The device (1) according to claim 1, wherein said means (7) for controlling the pressure (Pᵢ) of the pressurized air fed to said inlet (2a) comprises a feed pressure regulator (70).

6. The device (1) according to claim 1, further comprising an air cooler (90) for controlling the temperature (T) of the pressurized air fed to said inlet (2a) of said tube membrane (2).

7. The device (1) according to claim 1, wherein said control unit (100) is an electronic control unit connected to means (4) for monitoring at least one body parameter (VPₘ).

8. The device (1) according to claim 7, wherein said means (4) for monitoring at least one body parameter comprises at least a pulse oximeter (41).

9. The device (1) according to claim 1, further comprising at least one filtration unit (81) for purifying said pressurized air before feeding it to said inlet (2a).

10. A method of producing hypoxic and hyperoxic breathable gas mixtures with a device (1) according to claim 1, comprising the steps of feeding pressurized air to the membrane tube assembly (2) containing at least one hollow fiber (200), said membrane tube assembly (2) being providing a first flow of a hyperoxic gas mixture and a second flow of hypoxic gas mixture,
wherein the hypoxic gas mixture is a retentate gas mixture exiting said membrane tube assembly (2),
further comprising the step of regulating an oxygen content of said hyperoxic gas mixture and of said hypoxic gas mixture by controlling pressure and/or flow rate of said hypoxic gas mixture exiting from said membrane tube assembly (2) through said second retentate outlet (2c).

11. The method according to claim 10, wherein each gas mixture exiting said membrane tube assembly (2) is fed to a dispensing device (5) without being mixed with ambient air or with the other said gas mixture exiting the membrane tube assembly (2).

12. The method according to claim 10, wherein the oxygen content in said hyperoxic/hypoxic gas mixtures is a function of at least one of the pressure (Pᵢ) applied to the pressurized air injected into said tube membrane (2), the flow rate of the retentate gas mixture (F) defined at the second outlet (2c) of said membrane tube assembly (2) and the temperature (T) of the pressurized air fed to said tube membrane (2).

13. The method according to claim 10, wherein the oxygen content in said hypoxic and hyperoxic mixtures is controlled by means at least said means (6) for controlling the flow rate of the retentate gas mixture (F) at said second outlet (2c), which comprise a back pressure regulator (60) and/or a feed pressure regulator (70).

14. The method according to claim 10, wherein the oxygen percentage in said hyperoxic/hypoxic mixtures is controlled by means at least said means (7) for controlling the pressure (Pᵢ) of the pressurized air fed at said inlet (2a), which comprise a feed pressure regulator (70).

15. The method according to claim 10, wherein the oxygen percentage in said hyperoxic/hypoxic mixtures is controlled least by means (9) for controlling the temperature (T) of the pressurized air fed at said inlet (2a).

16. A method for operating a device (1) according to claim 1 comprising a dispensing device (5) and comprising means (4) for monitoring at least one body parameter (VPₘ), for performing intermittent hyperoxic/hypoxic training (IHHT), the method comprising the steps of:
continuously feeding pressurized air to said air inlet (2a) of said tube membrane (2),
detecting at least one body parameter (VPₘ) with said means (4) for monitoring at least one body parameter,
directly or indirectly calculating an oxygen saturation level (SpO₂) from said at least one detected body parameter (VPₘ) and comparing it with a maximum reference level (SpO_{2 r,max}) and with minimum reference level (SpO_{2 r,min}) stored in said control unit (100),
switching said valve means (21, 22) for alternatively providing to said dispensing device (5) said hyperoxic gas mixture in case said oxygen saturation level (SpO₂) is lower than said minimum reference level (SpO_{2 r,min}), or said hypoxic gas mixture in case said oxygen saturation level (SpO₂) is higher than said maximum reference level (SpO_{2 r,max}),
wherein said step of switching said valve means (21, 22) results in alternatively providing a hypoxic or a hyperoxic gas mixture to said dispensing device (5).

17. The method according to claim 16 wherein said maximum reference level of oxygen saturation (SpO_{2 r,max}) is a percentage value comprised between 98% and 100%, and wherein said minimum reference level of oxygen saturation (SpO_{2 r,min}) is a percentage value comprised between 84% and 86%.

18. The method according to claim 17 wherein said at least one body parameter (VPₘ) comprises oxygen saturation level (SpO₂) and/or a heart rate (BPM).

19. A breathable mixture as obtained by a method according to claim 10 with a device for providing hyperoxic/hypoxic breathable gas mixtures for use in medical treatment comprising:
at least one membrane tube assembly (2) for separating air into a permeate gas mixture and a retentate gas mixture, said membrane tube assembly (2) comprising a pressurized air inlet (2a), a first gas mixture outlet for permeate hyperoxic gas mixture (2b) and a second gas mixture outlet for retentate hypoxic gas mixture (2c), a source of pressurized air (3) for feeding pressurized air to said air inlet (2a), means (6) for controlling a flow rate (F) of said retentate gas mixture exiting said second outlet (2c) and means (7) for controlling pressure (Pᵢ) of the pressurized air fed to said inlet (2a),
a control unit (100) connected at least to said means (6) for controlling the flow rate (F) of said retentate gas mixture exiting said second outlet (2c) and to the means (7) for controlling the pressure (Pᵢ) of the pressurized air fed to said inlet (2a), and valve means (21, 22) for regulating said permeate and retentate gas mixtures flowing from said outlets (2b, 2c).

20. A method of treating stress-related and metabolic disorders that comprises administering an effective amount of an hypoxic and/or hyperoxic gas mixture as obtainable by a method according to claim 10.
